(19) United States
(12) Patent Application Publication
(10) Pub. No.: US 2023/0277164 A1
(43) Pub. Date: Sep. 7, 2023

(54) SAMPLE MANAGEMENT FOR CORE NEEDLE BIOPSY DEVICE

(71) Applicant: Devicor Medical Products, Inc., Cincinnati, OH (US)

(72) Inventors: Justin REBELLINO, Cincinnati, OH (US); Andrew P. NOCK, Dayton, OH (US); David C. McBREEN, West Chester, OH (US)

(21) Appl. No.: 18/109,532

(22) Filed: Feb. 14, 2023

Related U.S. Application Data
(60) Provisional application No. 63/316,184, filed on Mar. 3, 2022.
(60) Provisional application No. 63/340,030, filed on May 10, 2022.

(51) Int. Cl. A61B 10/02

(52) U.S. Cl. CPC A61B 10/0275

(57) ABSTRACT

A core needle biopsy device includes a needle assembly, a drive assembly, and a tissue sample holder. The needle assembly includes a piercer and a hollow cutter. The piercer includes a sharp distal tip and a notch proximal to the distal tip. The piercer is slidably disposed within the cutter to sever a tissue sample into the notch of the piercer. The drive assembly is configured to selectively move the piercer and the cutter. The tissue sample holder has a sample chamber, a drive member, and a wiper. The driver being configured to bend the wiper against a portion of the biopsy device to manipulate a severed tissue sample into the sample chamber.

### PRIORITY

This application claims priority to U.S. Provisional Application Ser. No. 63/316,184, entitled “Sample Management for Core Needle Biopsy Device,” filed on Mar. 3, 2022; and U.S. Provisional Application Ser. No. 63/340,030, entitled “Sample Cup for Core Needle Biopsy Device,” filed on May 10, 2022, the disclosures of which are incorporated by reference herein.

### BACKGROUND

A biopsy is the removal of a tissue sample from a patient to enable examination of the tissue for signs of cancer or other disorders. Tissue samples may be obtained in a variety of ways using various medical procedures involving a variety of the sample collection devices. For example, biopsies may be open procedures (surgically removing tissue after creating an incision) or percutaneous procedures (e.g. by fine needle aspiration, core needle biopsy, or vacuum assisted biopsy). After the tissue sample is collected, the tissue sample is typically analyzed at a lab (e.g. a pathology lab, biomedical lab, etc.) that is set up to perform the appropriate tests (such as histological analysis).

Biopsy samples have been obtained in a variety of ways in various medical procedures including open and percutaneous methods using a variety of devices. For instance, some biopsy devices may be fully operable by a user using a single hand, and with a single insertion, to capture one or more biopsy samples from a patient. In addition, some biopsy devices may be tethered to a vacuum module and/or control module, such as for communication of fluids (e.g., pressurized air, saline, atmospheric air, vacuum, etc.), for communication of power, and/or for communication of commands and the like. Other biopsy devices may be fully or at least partially operable without being tethered or otherwise connected with another device.

One technique for collecting a breast biopsy is to use a core needle biopsy device. One such device is the MAX-CORE disposable core biopsy instrument manufactured by Bard Biopsy Systems. Core needle biopsy devices frequently use a sharp, solid piercer equipped with a lateral tissue receiving notch positioned adjacent to the distal end of the piercer. When tissue is received within the notch, an elongate hollow cutting sheath is translated over the notch to sever a tissue sample. The severed tissue sample is then stored within the notch until both the piercer and the cutting sheath are removed from the patient. Thus, in core-needle biopsy devices, only one tissue sample can be collected per insertion of the piercer and cutting sheath.

In contrast to core needle breast biopsy procedures, vacuum-assisted breast biopsy devices permit the probe to remove multiple samples without requiring the probe be removed from the breast after every sample is collected. For instance, in a vacuum assisted breast biopsy device, a hollow needle is used to penetrate tissue. The hollow needle includes a lateral aperture adjacent to a sharp distal tip. A hollow cutter is disposed within the hollow needle and is moved axially relative to the lateral aperture of the needle to sever tissue samples. Once a tissue sample is severed by the hollow cutter, the tissue sample is transported axially though the cutter and collected in a tissue collection feature.

Examples of vacuum assisted biopsy devices and biopsy system components are disclosed in U.S. Pat. No. 5,526,822, entitled “Method and Apparatus for Automated Biopsy and Collection of Soft Tissue,” issued Jun. 18, 1996; U.S. Pat. No. 6,086,544, entitled “Control Apparatus for an Automated Surgical Biopsy Device,” issued Jul. 11, 2000; U.S. Pat. No. 6,162,187, entitled “Fluid Collection Apparatus for a Surgical Device,” issued Dec. 19, 2000; U.S. Pat. No. 6,432,065, entitled “Method for Using a Surgical Biopsy System with Remote Control for Selecting an Operational Mode,” issued Aug. 13, 2002; U.S. Pat. No. 6,752,768, entitled “Surgical Biopsy System with Remote Control for Selecting an Operational Mode,” issued Jun. 22, 2004; U.S. Pat. No. 7,442,171, entitled “Remote Thumbwheel for a Surgical Biopsy Device,” issued Oct. 8, 2008; U.S. Pat. No. 7,854,706, entitled “Clutch and Valving System for Tetherless Biopsy Device,” issued Dec. 1, 2010; U.S. Pat. No. 7,914,464, entitled “Surgical Biopsy System with Remote Control for Selecting an Operational Mode,” issued Mar. 29, 2011; U.S. Pat. No. 7,938,786, entitled “Vacuum Timing Algorithm for Biopsy Device,” issued May 10, 2011; U.S. Pat. No. 8,083,687, entitled “Tissue Biopsy Device with Rotatably Linked Thumbwheel and Tissue Sample Holder,” issued Dec. 21, 2011; U.S. Pat. No. 8,118,755, entitled “Biopsy Sample Storage,” issued Feb. 1, 2012; U.S. Pat. No. 8,206,316, entitled “Tetherless Biopsy Device with Reusable Portion,” issued on Jun. 26, 2012; U.S. Pat. No. 8,702,623, entitled “Biopsy Device with Discrete Tissue Chambers,” issued on Apr. 22, 2014; U.S. Pat. No. 8,858,465, entitled “Biopsy Device with Motorized Needle Firing,” issued Oct. 14, 2014; and U.S. Pat. No. 9,326,755, entitled “Biopsy Device Tissue Sample Holder with Bulk Chamber and Pathology Chamber,” issued May 3, 2016. The disclosure of each of the above-cited U.S. patents is incorporated by reference herein.

Additional examples of vacuum assisted biopsy devices and biopsy system components are disclosed in U.S. Pub. No. 2006/0074345, entitled “Biopsy Apparatus and Method,” published Apr. 6, 2006 and now abandoned; U.S. Pub. No. 2009/0131821, entitled “Graphical User Interface for Biopsy System Control Module,” published May 21, 2009, now abandoned; U.S. Pub. No. 2010/0152610, entitled “Hand Actuated Tetherless Biopsy Device with Pistol Grip,” published Jun. 17, 2010, now abandoned; U.S. Pub. No. 2010/0160819, entitled “Biopsy Device with Central Thumbwheel,” published Jun. 24, 2010, now abandoned; and U.S. Pub. No. 2013/0324882, entitled “Control for Biopsy Device,” published Dec. 5, 2013. The disclosure of each of the above-cited U.S. Patent Application Publications is incorporated by reference herein.

Examples of core needle biopsy devices are disclosed in U.S. Pat. No. 5,560,373, entitled “Needle Core Biopsy Instrument with Durable or Disposable Cannula Assembly,” issued on Oct. 1, 1996; U.S. Pat. No. 5,817,033, entitled “Needle Core Biopsy Device,” issued on Oct. 6, 1998; U.S. Pat. No. 5,971,939, entitled “Needle Core Biopsy Device,” issued on Oct. 26, 1999; and U.S. Pat. No. 5,511,556, entitled “Needle Core Biopsy Instrument,” issued on Apr. 30, 1996. The disclosure of each of the above-cited U.S. patents is incorporated by reference herein.

In some examples, it may be desirable to combine features from a core needle biopsy device and a vacuum assisted biopsy device to obtain the advantage of both devices and also reduce the overall disadvantages. For instance, core needle biopsy devices may be advantageous for their simplicity, light weight, and maneuverability. Furthermore, core needle biopsy devices generally include smaller sized needles, which can be desirable to increase patient comfort and recovery times. Meanwhile, vacuum assisted biopsy devices may be advantageous for their ability to collect multiple samples in a single insertion. Thus, a simple and light weight biopsy device capable of collecting multiple samples with a single insertion may be desirable.

One challenge in use of biopsy devices may include management of tissue samples once they are collected using the biopsy device. In some cases where a core needle biopsy device is used, challenges can arise due to the unique needle and cutter configuration. For instance, the cutter can be on the exterior of an inner piercer, stylet, or needle. A notch in the inner piercer can then be used to transport a severed tissue sample through the cutter. While the use of the notch can improve sample acquisition in some scenarios, collection of the severed tissue sample from the notch can be challenging due to the size and/or shape of the notch as well as the characteristics of the severed tissue sample (e.g., “sticky” or “clingy”). Thus, certain tissue sample collection features may be desirable for integration into a biopsy device that combines features of core needle biopsy devices and vacuum assisted biopsy devices.

While several systems and methods have been made and used for obtaining a biopsy sample, it is believed that no one prior to the inventors has made or used the invention described in the appended claims.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements. In the drawings some components or portions of components are shown in phantom as depicted by broken lines.

FIG. 1 depicts a perspective view of a version of a core needle biopsy device.

FIG. 2 depicts an exploded view of a needle assembly of the core needle biopsy device of FIG. 1.

FIG. 3 depicts a perspective view of the needle assembly of FIG. 2.

FIG. 4 depicts a perspective view of a drive assembly of the core needle biopsy device of FIG. 1.

FIG. 5 depicts a perspective view of a tissue sample holder of the core needle biopsy device of FIG. 1.

FIG. 6 depicts an exploded perspective view of the tissue sample holder of FIG. 5.

FIG. 7 depicts a perspective view of an outer housing of the tissue sample holder of FIG. 5.

FIG. 8 depicts an exploded perspective view of an extraction mechanism of the tissue sample holder of FIG. 5.

FIG. 9 depicts a perspective view of a wiper of the extraction mechanism of FIG. 8.

FIG. 10 depicts another perspective view of the tissue sample holder of FIG. 5, with the extraction mechanism positioned to collect a tissue sample.

FIG. 11A depicts cross-sectional end view of the tissue sample holder of FIG. 5, with the extraction mechanism positioned to collect a tissue sample.

FIG. 11B depicts another cross-sectional end view of the tissue sample holder of FIG. 5, with the extraction mechanism position to bend the wiper of FIG. 9.

FIG. 11C depicts still another cross-sectional end view of the tissue sample holder of FIG. 5, with the wiper of FIG. 9 propelling a tissue sample.

FIG. 11D depicts yet another cross-sectional end view of the tissue sample holder of FIG. 5, with a tissue sample propelling into a sample chamber of the tissue sample holder.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

Biopsy devices may be used to collect tissue samples in a variety of ways. For example, in some instances tissue samples are collected into a single tissue basket such that all tissue samples collected during a given biopsy procedure are deposited into the single tissue sample basket. In some other instances, tissue samples are collected into a tissue sample holder having separate compartments for each collected tissue sample. Such a multi-compartment tissue sample holder may additionally include trays or strips that individually hold each tissue sample separately from the other tissue samples. Such trays or strips may be removable or otherwise separable from the tissue sample holder at the conclusion of a biopsy procedure.

Regardless of the structure in which the tissue samples are stored, tissue samples may be collected using biopsy devices under the guidance of various imaging modalities such as ultrasound image guidance, stereotactic (X-ray) guidance, MRI guidance, Positron Emission Mammography (“PEM” guidance), Breast-Specific Gamma Imaging (“BSGI”) guidance, or otherwise. Each procedure has its own methodology based on the form of imaging guidance used.

Vacuum assisted biopsy devices and core needle biopsy devices both may have various advantages over the other, depending on context. For instance, one advantage of vacuum assisted biopsy devices is that vacuum assistance permits removal of multiple tissue samples using a single insertion. However, while core needle biopsy devices lack this feature, use of core needle biopsy devices may still be desirable in some scenarios. For instance, core needle biopsy devices may be generally capable of having smaller needles relative to vacuum assisted biopsy devices, thereby reducing patient anxiety and increasing the capacity of the needle to penetrate a lesion. Therefore, in some instances it may be desirable to incorporate the feature of multiple sample removal of a vacuum assisted biopsy device into a core needle biopsy device to achieve benefits present in both styles of biopsy device.

A desirable feature of the device described herein, which is a core needle biopsy device, is that the device allows for single insertion with multiple samples being obtained whilst using a core needle type device. To facilitate this functionality, the biopsy device further includes a tissue sample holder having one or more features to facilitate collection of a severed tissue sample from a notch, dugout, aperture, and/or other sample collection feature.

I. Example Core Needle Biopsy Device with Multi-Sample Collection

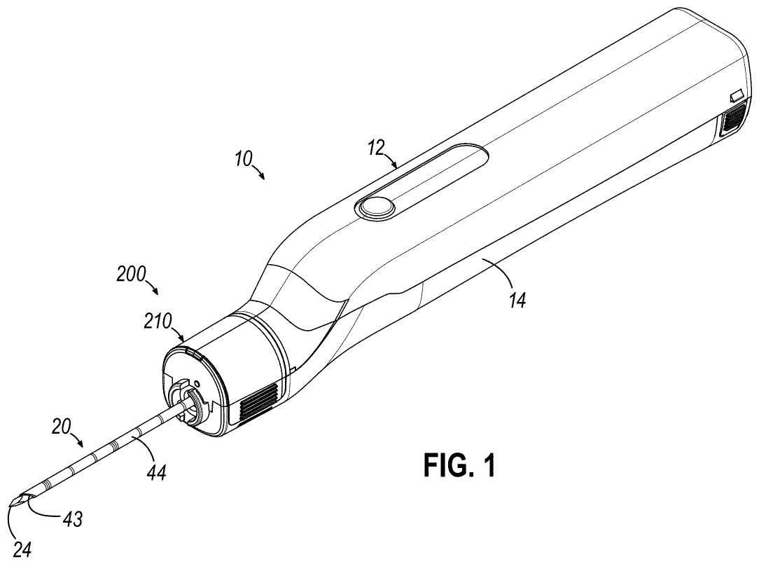

FIG. 1 shows a version of a core needle biopsy device (10) for use in a breast biopsy procedure. Core needle biopsy device (10) of the present version comprises a body (12) and a needle assembly (20) extending distally from body (12). Body (12) includes an outer housing (14) and an actuation member (16) disposed on outer housing (14). As will be describe in greater detail below, outer housing (14) encloses various components of biopsy device (10), which are used to drive needle assembly (20) through a cutting cycle and a tissue acquisition cycle. To this end, outer housing (14) of the present version is sized and shaped for grasping by an operator using a single hand. Although not shown, it should be understood that in some versions outer housing (14) may comprise multiple parts such that each part interconnects to form outer housing (14).

A. Example Needle Assembly

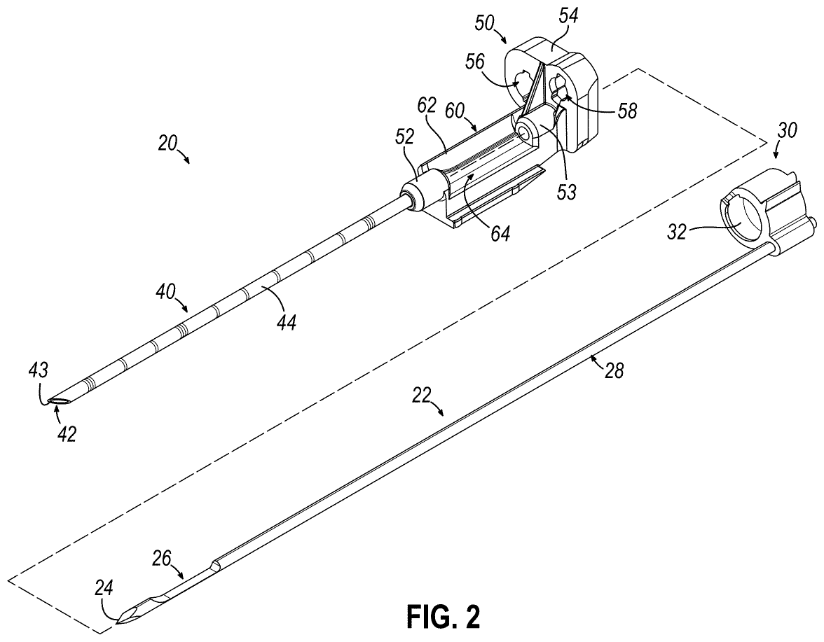

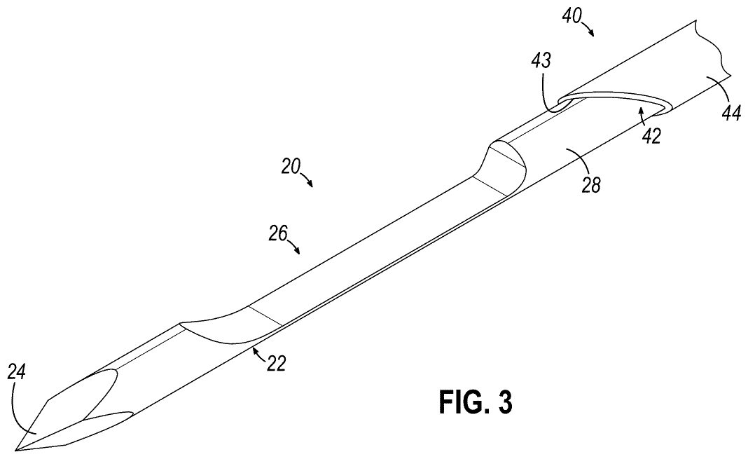

FIGS. 2 and 3 show needle assembly (20) in greater detail. As can be seen in FIG. 2, needle assembly (20) comprises an elongate piercer (22) and an elongate cutter (40). As will be described in greater detail below, piercer (22) is generally movable relative to cutter (40) to pierce tissue and collect tissue samples, while cutter is generally movable relative to piercer (22) to sever tissue samples. Piercer (22) comprises a generally cylindrical rod (28) (also referred to as a shaft) having a sharp distal tip (24) and a notch (26) disposed adjacent to distal tip (24). As will be described in greater detail below, distal tip (24) is generally configured to penetrate tissue of a patient. As will also be described in greater detail below, notch (26) is generally configured to receive tissue therein such that a tissue sample may be collected within notch (26) after the tissue sample is severed by cutter (40).

An end portion (30) is disposed on the proximal end of piercer (22). End portion (30) of the present version is overmolded, or otherwise fixedly secured to, the proximal end of piercer (22) and is generally configured to enhance the manipulability of piercer (22). In particular, end portion (30) comprises a receiving feature (32) in the form of a cylindrical recess. Receiving feature (32) is configured to receive a portion of a piercer drive assembly (130). Thus, receiving feature (32) in the present version may be configured as a carriage or other engagement feature configured to facilitate drive of piercer (22). As will be described in greater detail below, this permits piercer drive assembly (130) to drive movement of piercer (22) through a predetermined sequence of movement.

Cutter (40) comprises a generally hollow cylindrical tube that is configured to receive piercer (22) therein. Cutter (40) comprises an open distal end (42), a cannula portion (44) and an end portion (50). Open distal end (42) is configured to permit at least a portion of piercer (22) to protrude from cutter (40) when piercer (22) is moved relative to cutter (40). In some versions, such as the version shown, open distal end (42) may also be oriented at an angle relative to the longitudinal axis of cutter (40). In other versions, open distal end (42) may alternatively be perpendicular relative to the longitudinal axis of cutter (40). As will be described in greater detail below, this configuration permits needle assembly (20) to move through the cutting cycle and the tissue acquisition cycle by permitting notch (26) of piercer (22) to move relative to distal end (42) of cutter (40).

Open distal end (42) of the present version includes a tapered edge (43). Tapered edge (43) is generally configured to slice through tissue to separate tissue samples when cutter (40) is moved relative to notch (26) of piercer (22). Thus, it should be understood that tapered edge (43) is generally configured to act a blade. Although the present version is described and shown as using a tapered configuration, it should be understood that in other versions various alternative configurations can be used. For instance, in some versions tapered edge (43) includes a plurality of serrations in addition or in alternative to the taper shown. In still other versions, tapered edge (43) can include any other additional or alternative cutting surface as will be apparent to those of ordinary skill in the art in view of the teachings herein.

Cannula portion (44) of cutter (40) extends proximally from distal end (42) through end portion (50) such that piercer (22) can be received with the proximal end of cutter (40). Unlike end portion (30) of piercer (22), end portion (50) of cutter (40) is generally elongate such that end portion (50) can accommodate additional features that will be described in greater detail below. In the present version, the distal extension of end portion (50) can be relative to outer housing (14) to permit a portion of end portion (50) to be accessible to an operator for tissue sample collection purposes. Various suitable tissue collection mechanisms associated with end portion (50) will be described in greater detail below.

End portion (50) of cutter (40) comprises a cutter collar (52), a piercer collar (53), drive feature (54) and a tissue collection feature (60) (also referred to as a ledge, primary ledge, wiper catch, or wiper engagement feature) disposed between the cutter collar (52) and the drive feature (54). Cutter collar (52) is generally configured to receive the proximal end of cutter (40) to fixedly secure cutter (40) to end portion (50). Additionally, cutter collar (52) is configured to promote access of piercer (22) to cutter (40). In this configuration, piercer (22) may be slidably disposed within end portion (50) and extend distally through cutter (40). As will be described in greater detail below, this may permit piercer (22) to slide within cutter (40) and end portion (50) proximally to retract notch (26) from the proximal end of cutter (40) and thereby expose notch (26) within tissue collection feature (60).

Piercer collar (53) extends distally from a portion of drive feature (54) and is generally configured to slidably receive a portion of piercer (22). In particular, piercer collar (53) is positioned along a common axis with cutter (40) and cutter collar (52) to align piercer (22) with cutter (40) along the same common axis. Thus, cutter (40), cutter collar (52), and piercer collar (53) may be configured to act cooperatively to maintain piercer (22) along a common axis. As will be described in greater detail below, this feature may be desirable during use to remove a tissue sample from notch (26) when notch is disposed within tissue collection feature (60).

Drive feature (54) of end portion (50) is generally configured to engage features of a cutter drive assembly (120) for manipulation of cutter (40) via drive end portion (50) through a predetermined sequence of movement. Although various suitable configurations may be used, in the present version, drive feature (54) includes a drive opening (56) and a release opening (58). Each opening (56, 58) may be configured to engage corresponding components of cuter drive assembly (120) to permit manipulation of cutter (40) via end portion (50).

Tissue collection feature (60) is disposed distally relative to drive feature (54). Tissue collection feature (60) generally defines an elongate notch that is open, or otherwise exposed, relative to cannula portion (44) of cutter (40). In the present version, the proximal end of cutter (40) is disposed distally of tissue collection feature (60) to expose the interior of cutter (40) relative to tissue collection feature (60). However, it should be understood that in other versions, cannula portion (44) may include a cutout, opening, lateral aperture, or other feature that may be adjacent to, or otherwise define, tissue collection feature (60). Regardless, it should be understood that tissue collection feature (60) is in communication with the hollow interior, or a lumen, defined by cannula portion (44). As will be described in greater detail below, this relationship between tissue collection feature (60) and cannula portion (44) permits an operator to remove tissue samples from cutter (40) as they are collected by piercer (22).

Tissue collection feature (60) includes an engagement ledge (62) (also referred to as a protrusion, primary ledge, stopping member, or tensioner) and a recessed portion (64). Engagement ledge (62) extends upwardly relative to cutter collar (52) and piercer collar (53), defining a curved or wave-shaped profile. Engagement ledge (62) further extends longitudinally along the length of tissue collection feature (60) or from cutter collar (52) to piercer collar (53). As will be described in greater detail below, engagement ledge (62) is generally configured to engage one or more portions of a tissue sample holder (200) to facilitate transfer of a tissue sample from notch (26) of piercer (22) into tissue sample holder (200).

Recessed portion (64) is positioned adjacent or proximate to engagement ledge (62) and extends from cutter collar (52) to piercer collar (53). Recessed portion (64) is generally configured to receive piercer (22) therein to provide access to notch (26) of piercer (22). As such, recessed portion (64) defines a curved shape that may be complementary to the shape of piercer (22).

FIG. 3 shows piercer (22) disposed within cutter (40). As can be seen, cutter (40) is generally configured to receive piercer (22) such that piercer (22) is coaxial with cutter (40). In addition, piercer (22) is generally movable relative to open distal end (42) of cutter (40). It should be understood that in some circumstances piercer (22) moves relative to cutter (40), while cutter (40) remains stationary. In other circumstances, cutter (40) moves relative to piercer (22), while piercer (22) remains stationary. In either case, it should be understood that piercer (22) and cutter (40) are generally configured such that notch (26) of piercer (22) moves into and out of cutter (40) such that notch (26) can be disposed distally or proximally relative to open distal end (42) of cutter (40). As will be described in greater detail below, this configuration permits piercer (22) and cutter (40) to operate cooperatively to pierce tissue, cut a tissue sample, and retract the tissue sample for collection by an operator via tissue collection feature (60).

B. Example Drive Assembly

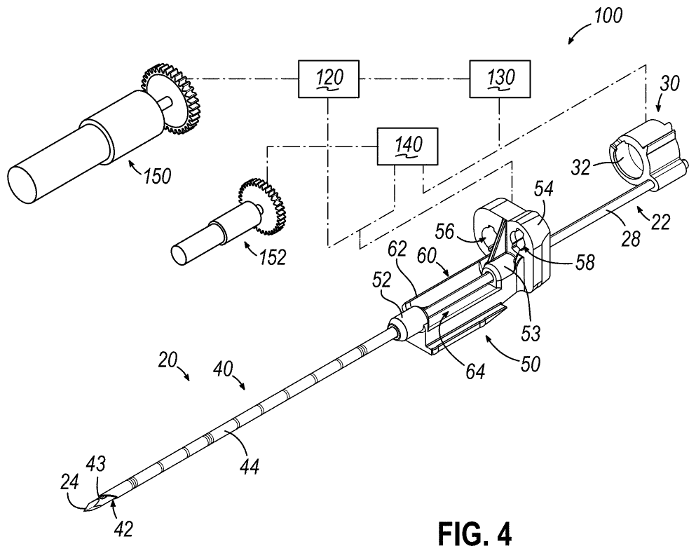

FIG. 4 shows the internal components of body (12) of biopsy device (10) with outer housing (14) removed. As can be seen, inside outer housing (14), body (12) includes a drive assembly (100). Drive assembly (100) is generally configured to engage needle assembly (20) to drive piercer (22) and cutter (40) through a predetermined sequence of movements to thereby pierce tissue and acquire a plurality of tissue samples with a single insertion of needle assembly (20) into a patient. Although not shown, it should be understood that outer housing (14) defines various internal geometries that support or otherwise engage drive assembly (100). As will be understood, such internal geometries are used to provide relative movement of various components of drive assembly (100) relative to other components of drive assembly (100) and/or outer housing (14).

Drive assembly (100) comprises a cutter drive assembly (120), a piercer drive assembly (130), and a firing assembly (140). Generally, needle firing assembly (140) is configured to cock and fire cutter (40) and piercer (22) in a predetermined sequence to sever a tissue sample. To collect the severed tissue sample, cutter drive assembly (120) is generally configured to retract cutter (40). Similarly, piercer drive assembly (130) is generally configured to retract piercer (22). It should be understood that, in some versions, both cutter drive assembly (120) and piercer drive assembly (130) can be configured to rotate cutter (40) and/or piercer (22), respectively.

Needle firing assembly (140) is generally shown schematically in the present version. Thus, it should be understood that in some versions needle firing assembly (140) can take on a variety of forms having a combination of gears, racks, leadscrews, carriages, springs, and/or etc. Such components of needle firing assembly (140) can generally be configured to rapidly fire cutter (40) and piercer (22) in a predetermined sequence to penetrate tissue. For instance, in some versions, needle firing assembly (140) is configured to rapidly fire piercer (22) distally to penetrate tissue. Needle firing assembly (140) may also configured to rapidly fire cutter (40) distally. The firing of cutter (40) can be either delayed relative to piercer (22) or slower relative to piercer (22) such that notch (26) can be exposed relative to cutter (40). This sequence can permit tissue to enter notch (26), so that it can be severed by subsequent movement of cutter (40). In addition, it should be understood that needle firing assembly (140) can include other components and/or features to permit cocking of cutter (40) and/or piercer (22) prior to firing.

Cutter drive assembly (120) is generally configured to translate and/or rotate cutter (40) either independently of piercer (22) or in concert therewith. For instance, cutter drive assembly (120) can include various combinations gears, racks, leadscrews, carriages, springs, and/or etc. to drive cutter (40) through a predetermined sequence. In one such sequence, cutter (40) is retracted proximally relative to outer housing (14) to prepare cutter (40) for a tissue collection sequence that will be described in additional detail below. In addition, cutter drive assembly (120) can also be configured to rotate cutter (40) in a predetermined sequence to assist with the tissue collection sequence described in greater detail below.

Piercer drive assembly (130) is generally configured to translate and/or rotate piercer (22) either independently of cutter (40) or in concert therewith. For instance, piercer drive assembly (120) can include various combinations of gears, racks, leadscrews, carriages, springs, and/or etc. to drive piercer (22) through a predetermined sequence. In one such sequence, piercer (22) is retracted proximally relative to cutter (40) after severing a tissue sample to retract the tissue sample proximally towards outer housing (14). Once piercer (22) is retracted, the tissue sample can be extracted for collection in the tissue collection sequence described in greater detail below.

In the present version, drive assembly (100) is powered by one or more motors (150, 152). In particular, drive assembly (100) of the present version includes a drive motor (150) and a firing motor (152). Drive motor (150) of the present version is in communication with both cutter drive assembly (120) and piercer drive assembly (130) to provide rotational motion to both assemblies, which ultimately drives translation and/or rotation of both cutter (40) and piercer (22). Similarly, firing motor (152) is in communication with firing assembly (140) to drive firing and/or cocking of cutter (40) and piercer (22). Although drive assembly (100) of the present version includes two motors (150, 152), it should be understood that in other versions any suitable number of motors may be used such as a single motor, or three or more motors. In addition, motors (150, 152) can be configured to drive cutter drive assembly (120), piercer drive assembly (130), and/or firing assembly (140) in various combinations.

Although cutter drive assembly (120), piercer drive assembly (130), and firing assembly (140) of the present version are shown schematically as three separate drive assemblies, it should be understood that in other versions various elements of cutter drive assembly (120), piercer drive assembly (130), and firing assembly (140) can be combined into a single drive assembly or multiple drive assemblies to drive motion of cutter (40) and piercer (22) in accordance with the sequences described herein. In some versions, cutter drive assembly (120), piercer drive assembly (130), and firing assembly (140) can be constructed in accordance with at least some of the teachings of U.S. Ser. No. 16/381,573, entitled “Core Needle Biopsy Device for Collecting Multiple Samples in a Single Insertion,” filed on Apr. 11, 2019, the disclosure of which is incorporated by reference herein.

C. Example Tissue Sample Holder

As noted above, in the present version, needle assembly (20) is configured as a core needle-style tissue acquisition assembly that can collect multiple samples using a single insertion. In some versions, each time a tissue sample is collected, the tissue sample can be physically removed from tissue collection feature (54) by an operator and deposited in a separate location (e.g., formalin jar). However, this physical removal may be undesirable in some versions because it can add an additional step to the biopsy procedure, thereby increasing procedure times. Moreover, this physical removal can introduce extra variables into the biopsy procedure by requiring the operator to keep track of collected tissue samples throughout a biopsy procedure. This physical removal can also lead to frequent operator grip changes throughout a biopsy procedure, which may be generally undesirable. Accordingly, in some versions, it may be desirable to include a tissue sample holder or other sample collection mechanism within biopsy device (10) to collect and store tissue samples throughout a biopsy procedure.

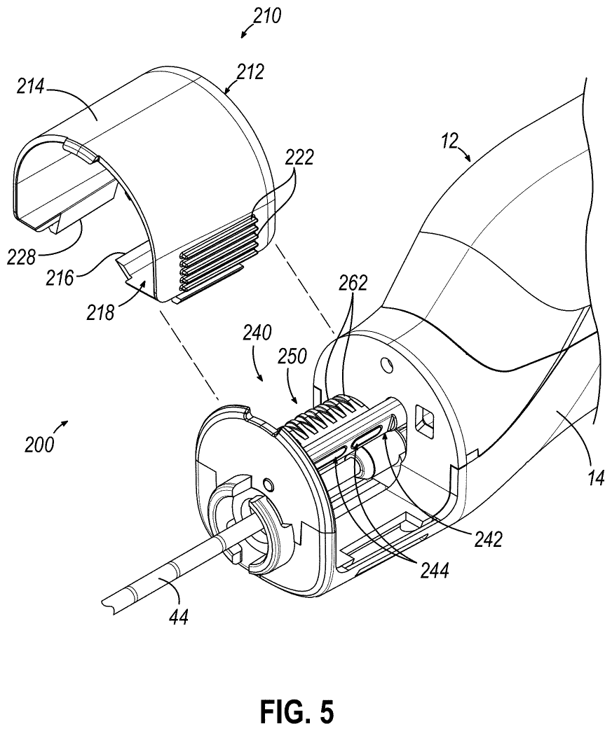

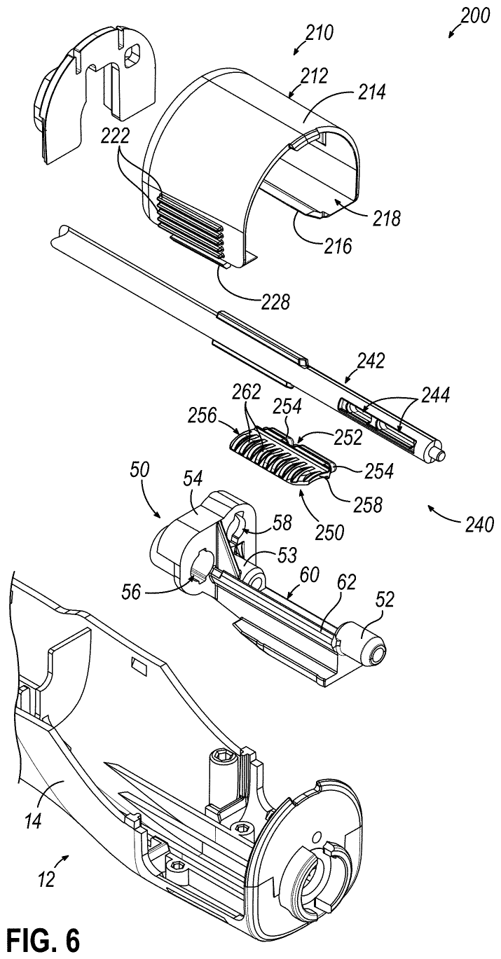

FIGS. 5 and 6 show a version of a tissue sample holder (200) that can be readily incorporated into biopsy device (10) described above. Tissue sample holder (200) of the present version includes an extraction mechanism (240) disposed within a generally cylindrical outer housing (210). Tissue sample holder (200) is generally configured to collect a plurality of tissue samples from tissue collection feature (60) of needle assembly (20) during a biopsy procedure using rotation of extraction mechanism (240). As will be described in greater detail below, tissue sample holder (200) is generally configured to collect and store tissue samples in a bulk collection configuration of any suitable number.

Outer housing (210) is generally configured to enclose extraction mechanism (240) within a chamber defined by outer housing (210). In the present version, outer housing (210) is formed of a combination of elements, although in other versions a single element may be used. Specifically, outer housing (210) of the present version includes a removable cover (212) (also referred to as a cup, sample cup, sample window, sample receiver, or sample accumulator) that is configured to fasten to a portion of body (12) of biopsy device (10) configured to receive cover (212). Thus, the combination of cover (212) and body (12) of biopsy device (10) together define outer housing (210). In the present version, cover (212) and body (12) may together seal in interior of tissue sample holder (200) from the external environment. Thus, in some versions, cover (212) and or body (12) may include certain sealing elements to promote a fluid and/or air-tight seal between cover (212) and body (12).

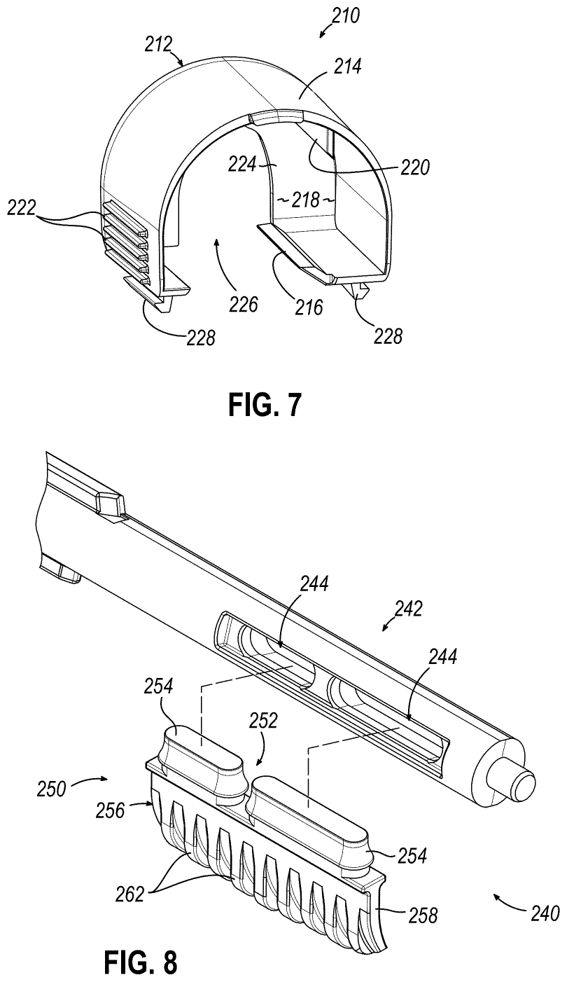

Cover (212) is shown in greater detail in FIG. 7. Cover (212) is generally configured to facilitate collection of one or more tissue samples through engagement with extraction mechanism (240), as will be described in greater detail below. Although features of cover (212) to facilitate collection of one or more tissue samples are described herein as being integral with cover (212), it should be understood that in other versions, such features may be separate components from cover (212) either secured to cover (212) or other portions of biopsy device (10).

As best seen in FIG. 7, cover (212) includes a generally D-shaped outer wall (214). Outer wall (214) is configured to cover at least a portion of body (12) of body device (10) to enclose at least a portion of extraction mechanism (240) within the interior defined by outer wall (214). Outer wall (214) includes various features projecting from portions of outer wall (214) to facilitate function of tissue sample holder (200). For instance, outer wall (214) defines a piercer interface (216), a sample chamber (218), and an axillary sample catch (220) (also referred to as a sample knife, secondary catch, secondary ledge, or sample scraper). Piercer interface (216), sample chamber (218) and sample catch (220) may generally operate cooperatively with extraction mechanism (240) to collect and store one or more tissue samples obtained via needle assembly (20).

Piercer interface (216) projects inwardly (e.g., upwardly, laterally, or a combination of both) from a portion of outer wall (214) at an angle. Piercer interface (216) defines a slight ledge, shelf, or overhang. As will be described below, the ledge defined by piercer interface (216) may be positioned proximate a portion of end portion (50) to direct one or more tissue samples towards sample chamber (218). Additionally, the angled projection of piercer interface (216) separates sample chamber (218) from the rest of the interior of outer wall (214) to at least partially define a discrete area for the collection of one or more tissue samples.

Sample chamber (218) is generally configured to receive a plurality of tissue samples. As described above, at least a portion of sample chamber (218) is defined by piercer interface (216). Specifically, piercer interface (216) in the present version defines one side of sample chamber (218). Meanwhile an opposite side and the floor of sample chamber (218) is defined by outer wall (214). Specifically, the curved portion of outer wall (214) defines the side of sample chamber (218) opposite piercer interface (216) and the floor is defined by a lateral extension of outer wall (214). These features together define a recessed portion within outer wall (214) that defines sample chamber (218).

Sample catch (220) extends inwardly from an upper portion of outer wall (214) defining projection into the interior of outer wall (214). As will be described in greater detail below, sample catch (220) is generally configured to engage one or more portions of extraction mechanism (240) to transfer a tissue sample onto the surface of sample catch (220). To facilitate such functionality, sample catch (220) may have a variety of configurations. For instance, in the present version sample catch (220) is configured as a rectangular member extending from a distal end of outer wall (214) to a proximal end of outer wall (214). Optionally, the inner most end of sample catch (220) may be angled in some versions to provide an edge that may be used to scrape or otherwise remove a tissue sample from a portion of extraction mechanism (240). Although sample catch (220) of the present version is generally rectangular, in other versions, sample catch (220) may take on a variety of alternative shapes such as curved. In addition, sample catch (220) in some versions may include multiple teeth or extension members rather than being a single continuous part as shown.

Cover (212) further includes certain features configured to promote engagement of cover (212) with body (12) of biopsy device (10). Specifically, cover (212) includes a grip (222), a proximal wall (224), and one or more engagement members (228). Grip (222) is configured to enhance an operators grip on cover (212) during removal of cover (212) from body (12). As such, grip (222) of the present version includes a plurality of bumps or ribs projecting from an outer surface of outer wall (214). Of course, in other versions, grip (222) may include other grip-promoting structures such as indentations, knurling, and/or etc.

Proximal wall (224) extends laterally across the proximal end of outer wall (214). Proximal wall (224) is generally configured to enclose the chamber defined by cover (212) in cooperation with body (12). In some versions, proximal wall (224) may be a separate component that may be removable from cover (212). To promote access to the interior of cover (212), proximal wall further includes an access opening (226). Access opening (226) is generally configured to correspond to the shape of various structures that may extend into or through cover (212). For instance, in the present version, cutter (40) and end portion (50) both may extend into or through a portion of cover (212). Thus, access opening (224) is sized and shaped to accommodate both cutter (40) and at least a portion of end portion (50).

Engagement members (228) extend from a bottom surface of outer wall (214). Engagement members (228) are generally configured to engage at least a portion of body (12) to releasably secure cover (212) to body (12). For instance, in some versions, body (12) may include a notch, recess, indentation, depression, and/or etc. that may be covered or enclosed by cover (212). Such a notch, recess, indentation, depression or other feature may expose a portion of needle assembly (20) therein such that a portion of needle assembly (20) may be visible through at least a portion of cover (212). Engagement members (228) may thus facilitate coupling of cover (212) to body (12) to promote the enclosure of a portion of body (12) by cover (212). In the present version, engagement members (228) include snap-fit fasteners that may be configured to snap into engagement with corresponding features of body (12). Of course, various alternative fastening mechanisms may be used.

As best seen in FIG. 8, extraction mechanism (240) includes a driver (242) (also referred to as a rotatable member, drive shaft, or actuation member) and a wiper (250) (also referred to as a sample blade, or sample manipulator). Driver (242) of the present version is configured as an elongate shaft that is rotatable within the interior of outer housing (210). Specifically, driver (242) may be configured to rotate a full 360° or more to facilitate the collection of multiple tissue samples. In some versions, driver (242) may extend proximally into body (12) and may be in communication with drive assembly (100) such that driver (242) may be driven with drive assembly (100). In such versions, movement of driver (242) may be in a predetermined sequence with movement of piercer (22) and cutter (40). Additionally, driver (242) may be configured in some versions to drive movement of other components of biopsy device (10). For instance, in some versions, driver (242) may be used to release end portion (50) of cutter (40) to facilitate firing of cutter (40). Of course, other suitable uses for driver (242) will be apparent to those of ordinary skill in the art in view of the teachings herein.

Driver (242) includes one or more openings (244) extending laterally through driver (242). Openings (244) are generally configured to receive at least a portion of wiper (250) to permit coupling of wiper (250) to driver (242). As such, openings (244) are positioned proximate the distal end of driver (242) at a point that aligns with other components of tissue sample holder (200) such as piercer interface (216), sample chamber (218) and/or sample catch (220).

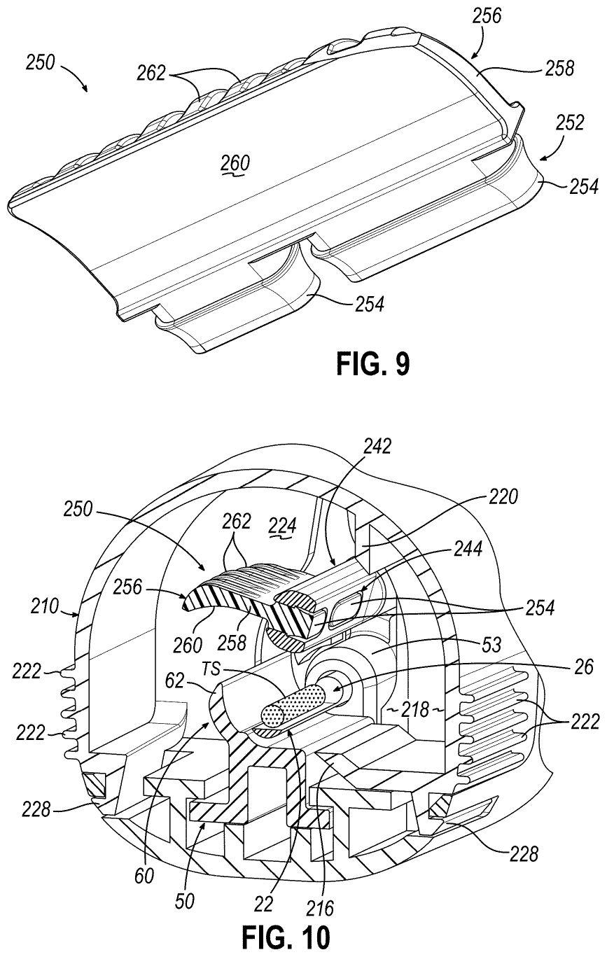

As best seen in FIGS. 8 and 9, wiper (250) is configured to couple to openings (244) of driver (242) so that driver (242) may rotate wiper (250) to remove a tissue sample from notch (26) of piercer (22) and move the tissue sample into sample chamber (218) of tissue sample holder (200). As will be understood, wiper (250) is generally configured to operate cooperatively with other portions of tissue sample holder (200) to propel the tissue sample as a projectile into sample chamber (218). This propulsion of the tissue sample may be characterized in a variety of ways such as slapping, flicking, snapping, etc. Although the present version includes a single wiper (250), it should be understood that in other versions multiple wipers (250) may be used.

Wiper (250) comprises an attachment portion (252), and a manipulation portion (256). Attachment portion (252) includes one or more connectors (254) configured to engage a corresponding opening (244) of driver (242). In the present version, connectors (254) are each generally configured as a mushroom connector or other interference fit-type feature to secure wiper (250) to driver (242). Of course, in other versions, various alternative configurations for connectors (254) may be used. In such alternative configurations, an interference fit may be used. Alternatively, in other configurations a snap-fit may be used. In still other configurations, permanent fastenings may be used such as overmolding or adhesive bonding.

Manipulation portion (256) includes a curved body (258) with an engagement surface (260) on one side of curved body (258) and a plurality of ribs (262) (also referred to as fins) on another opposite side of curved body (258). The curvature of curved body (258) is generally in the direction of rotation provided by driver (242). In other words, curved body (258) extends laterally away from driver (242) while also curving away from the longitudinal axis of driver (242). Although the term “curved” is used in connection with curved body (258) herein, it should be understood that in some versions, curved body (258) may optionally extend straight from driver (242) and not be curved. As will be described in greater detail below, the curvature of curved body (258) promotes engagement with other portions of tissue sample holder (200) in a specific predetermined sequence to facilitate manipulation of tissue samples.

Engagement surface (260) is generally flat and smooth. The orientation of engagement surface (260) is on the inside of curved body (258) such that engagement surface (260) defines a slight concavity. Engagement surface (260) is configured to contact a tissue sample to move the tissue sample from notch (26) of piercer (22) to sample chamber (218). In some versions, engagement surface (260) may additionally include certain features to prevent adhesion or sticking between engagement surface (260) and a tissue sample. For instance, in some versions, engagement surface (260) (or all external surfaces of wiper (250)) may include a hydrophobic or non-stick coating to prevent sticking of the tissue sample to engagement surface (260). In addition, or in the alternative, some versions of engagement surface (260) may include bumps or other non-smooth surface features that may reduce the propensity of tissue to stick to engagement surface (260). In such examples, the presence of bumps may reduce the need for coatings. In still other versions, engagement surface (260) may include a hydrophilic coating.

Ribs (262) extend laterally from attachment portion (252) on a surface of curved body (258) opposite of engagement surface (260). The thickness of each rib (262) generally increases as each rib (262) extends from attachment portion (252). This increase in thickness provides an increased amount of material of wiper (250) in an area laterally offset from attachment portion (252). This increased amount of material is generally desirable to change the center of mass of wiper (250) and thereby promote enhanced weight distribution. As noted above and will be described in greater detail below, wiper (250) is generally configured to propel, slap or flick a tissue sample. As such, it may be desirable to have a concentration of mass towards the lateral end of wiper (250) to promote the desired manipulation of a tissue sample. Furthermore, in some versions, the structure of ribs (262) may be used to promote at least some rigidity within the structure of wiper (250).

All ribs (262) in the present version are generally of a uniform width (relative to the axis of driver (242)) and spacing relative to each other. It should be understood that in some versions, the width and/or spacing of one or more individual ribs (262) may be modified to influence the balance characteristics of wiper (250). For instance, in some versions, one or more ribs (262) may be wider than other ribs (262), or some ribs (262) may be more compactly spaced than other ribs (262) to concentrate mass of ribs (262) in a particular region of wiper (250). By way of example only, in some versions, a rib (262) on the proximal end of wiper (250) may have a width about 3.5 times greater or more than the other ribs (262) to concentrate mass towards the proximal end of wiper (250).

Although wiper (250) of the present example is of a single piece of material, it should be understood that in other versions, wiper (250) may be divided in one or more ways. For instance, in some versions, wiper (250) may include one or more slots, which may be used to divide engagement surface (260) into different sections with each section being configured to flex independently of other sections. Such slots may extend inwardly from the outer edge of wiper (250) toward attachment portion (252). In such versions, the particular length of each slot may be 25 to 75% of the width of wiper (250). In still other versions, such slots may extend the full width of wiper (250), dividing wiper (250) entirely. In other words, in some versions, wiper (250) may be configured as a plurality of separate wipers (250) connected to driver (254) in various positions along the length of driver (254).

In the present version, wiper (250) comprises a material having specific properties that provide a balance between flexibility and rigidity. Examples of materials having a suitable balance between flexibility and rigidity may include, for example, polymers or rubbers. In some versions, regardless of the particular material used, this balance may be characterized as a durometer. Various suitable durometers may be used. For instance, in the present version, a durometer of 85 may be used. In other versions, a durometer of 76.5 to 93.5 may be used. Of course, in other versions, various suitable durometers may be used as will be apparent to those of ordinary skill in the art in view of the teachings herein.

FIG. 10 shows the relationship between extraction mechanism (240), outer housing (210), end portion (50) of cutter (40) and piercer (22) when biopsy device (10) is configured for extraction of a tissue sample from notch (26) of piercer (22). In this configuration, piercer (22) is retracted proximally to align notch (26) with tissue collection feature (60) of end portion (50). Meanwhile, end portion (50) is advanced distally such that end portion (50) is disposed within outer housing (210). Specifically, tissue collection feature (60) of end portion (50) is aligned with both piercer interface (216) of outer housing (210) and wiper (250) of extraction mechanism (240). In this alignment, tissue collection feature (60) defines a curved pathway extending towards notch (26) of piercer (22) and into sample chamber (218). As such, wiper (250) may sweep along the curved pathway defined by tissue collection feature (60) to transport a tissue sample from notch (26) and into sample chamber (218).

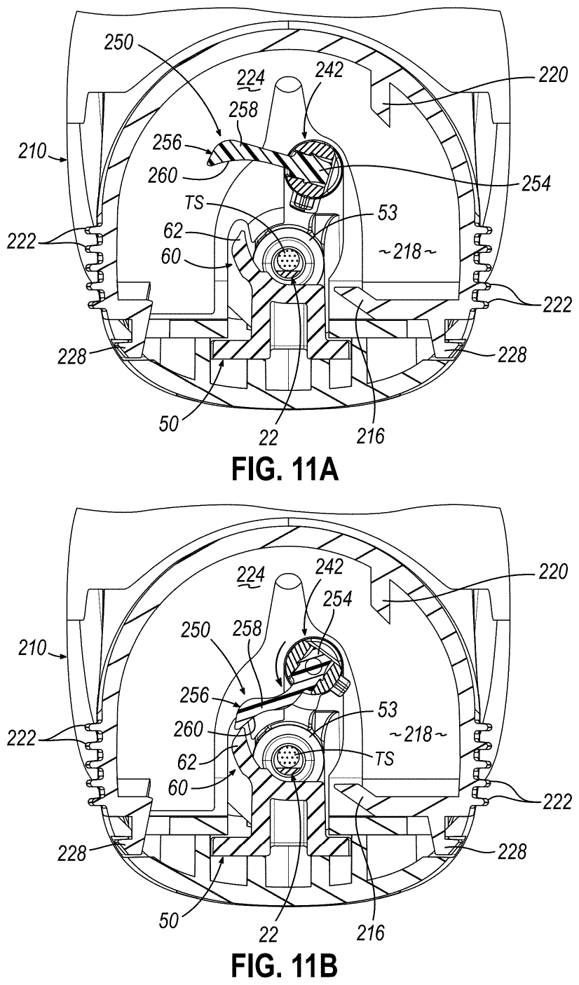

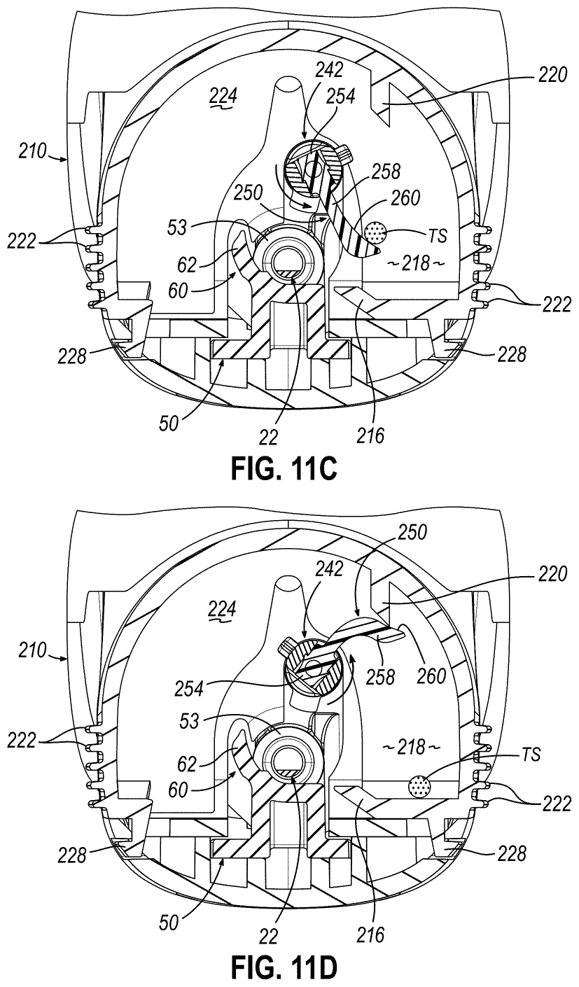

FIGS. 11A through 11D show an example of a use of wiper (250) to transport a tissue sample from notch (26) into sample chamber (218). As can be seen in FIG. 11A, piercer (22) and cutter (40) may initially begin in the position described above where biopsy device (10) is configured for extraction of a tissue sample. This corresponds to notch (26) of piercer (22) being retracted to align with tissue collection feature (60) of end portion (50) to expose a tissue sample to the interior of tissue sample holder (200). At this stage, driver (242) of extraction mechanism (240) is rotated to position wiper (250) at about a 9 o'clock position so that wiper is positioned slightly above tissue collection feature (60).

Once biopsy device (10) is configured for extraction of a tissue sample as described above and shown in FIG. 11A, extraction mechanism (240) can be used to extract a tissue sample. To extract a tissue sample, driver (242) may rotate in a counterclockwise direction (viewed from the distal end of biopsy device (10) toward the proximal end of biopsy device (10)) to move a portion of wiper (250) into contact with tissue collection feature (60) of end portion (50). As shown in FIG. 11B, this rotation of driver (242) may continue once engagement between wiper (250) and tissue collection feature (60) is achieved to apply tension to wiper (250) due to an interference between wiper (250) and tissue collection feature (60). This application of tension to wiper (250) may result in some bending of wiper (250) as shown in FIG. 11B. Thus, driver (242) may be configured to bend wiper (250) against a portion of biopsy device (10). Regardless, the applied tension may store at least some potential energy within wiper (250) as driver (242) continues to rotate while a portion of wiper (250) is held in a fixed position by engagement with tissue collection feature (60).

Upon further rotation of driver (242), wiper (250) may continue to bend until wiper (250) is pulled past a portion of tissue collection feature (60). Once wiper (250) is pulled past the portion of tissue collection feature (60), wiper (250) may release suddenly from engagement with tissue collection feature (60), thereby releasing potential energy stored within wiper (250). During this release of stored potential energy, wiper (250) may rapidly advance around the curvature defined by tissue collection feature (60) and into notch (26) of piercer (22) and into engagement with a tissue sample (TS) within notch (26). Rapid movement of wiper (250) may then continue with the tissue sample (TS) disposed on engagement surface (260) of wiper (250), thereby propelling the tissue sample (TS) out of notch (26) and towards sample chamber (218) as shown in FIG. 11C.

As wiper (250) consumes the stored potential energy, movement of wiper (250) may rapidly slow (or even reverse in some circumstances) to approximately match that of driver (242). As movement of wiper (250) slows, momentum of the tissue sample (TS) may continue in the initial direction of wiper (250). As such, the tissue sample (TS) may separate from wiper (250) at this stage and be propelled from wiper (250) towards sample chamber (218) as shown in FIG. 11D. In addition, or in the alternative, sample catch (220) may contribute to the sudden slowing of wiper (250) to propel the tissue sample (TS). In either case, the release of potential energy from wiper (250) generally may result in the tissue sample (TS) being flung, slapped, or shot from notch (26) of piercer (22) and into sample chamber (218).

In some scenarios, wiper (250) and the tissue sample (TS) may fail to separate for a variety of reasons. For instance, in some circumstances, the tissue sample (TS) may be atypically dry or atypically wet. Consequently, the tissue sample (TS) may be more tacky or sticky and may therefore have the propensity to stick to wiper (250). In other circumstances, the tissue sample (TS) may be less dense than a more common tissue sample (TS). Consequently, the tissue sample (TS) may have less momentum leading to separation from wiper (250). Regardless of the circumstances, in scenarios where separation fails, sample catch (220) may act as a secondary separation device to scrape or otherwise remove the tissue sample (TS) from wiper (250) as wiper (250) is rotated past sample catch (220).

Once the tissue sample (TS) has been successfully separated from wiper (250) and deposited in sample chamber (218), driver (242) may continue to rotate to return wiper (250) to the position shown in FIG. 11A. Piercer (22) and cutter (40) may then be used to sever and collect another tissue sample and the process described above with respect to FIGS. 11A through 11D may be repeated to collect one or more subsequent tissue samples within sample chamber (218). The process may then be repeated until a desired number of tissue samples have been collected.

II. Example Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

A core needle biopsy device, comprising: a needle assembly, wherein the needle assembly includes a piercer and a hollow cutter, wherein the piercer includes a sharp distal tip and a notch proximal to the distal tip, wherein the piercer is slidably disposed within the cutter to sever a tissue sample into the notch of the piercer; a drive assembly configured to selectively move the piercer and the cutter; and a tissue sample holder having a sample chamber, a driver, and a wiper, the driver being configured to bend the wiper against a portion of the biopsy device to manipulate a severed tissue sample into the sample chamber.

### Example 2

The core needle biopsy device of Example 1, the driver including a rotatable shaft, the wiper being secured to the shaft such that rotation of the shaft is configured to rotate the wiper relative to the piercer to thereby manipulate a severed tissue sample into the sample chamber.

### Example 3

The core needle biopsy device of Examples 1 or 2, the needle assembly further comprising a cutter end portion associated with a proximal end of the cutter, the cutter end portion including a ledge configured to engage the wiper.

### Example 4

The core needle biopsy device of Example 3, the cutter end portion including a curved surface extending towards a portion of the piercer.

### Example 5

The core needle biopsy device of any one or more of Examples 1 through 4, the tissue sample holder further including an outer cover configured to removably couple to a housing of the biopsy device, the outer cover defining at least a portion of the sample chamber.

### Example 6

The core needle biopsy device of Example 5, the outer cover including one or more wall defining the sample chamber.

### Example 7

The core needle biopsy device of Examples 5 or 6, the outer cover including a piercer interface positioned proximate a portion of the piercer.

### Example 8

The core needle biopsy device of any one or more of Examples 5 through 7, the outer cover including a sample catch, the sample catch being configured to engage a portion of the wiper to scrape a tissue sample from the wiper.

### Example 9

The core needle biopsy device of Example 8, the sample catch being positioned proximate the sample chamber.

### Example 10

The core needle biopsy device of any one or more of Examples 5 through 9, the outer cover including one or more connectors configured to releasably secure the outer cover to the housing of the biopsy device.

### Example 11

The core needle biopsy device of any one or more of Examples 1 through 10, the tissue sample holder including a single wiper.

### Example 12

The core needle biopsy device of any one or more of Examples 1 through 11, the wiper including a material having a durometer of 76.5 to 93.5.

### Example 13

The core needle biopsy device of any one or more of Examples 1 through 12, the wiper including a material having a durometer of about 85.

### Example 14

The core needle biopsy device of any one or more of Examples 1 through 13, the wiper having a hydrophobic coating.

### Example 15

The core needle biopsy device of any one or more of Examples 1 through 14, further comprising a body having a distal end, the needle assembly extending distally from the distal end of the body, the tissue sample holder being disposed on the distal end of the body.

### Example 16

A tissue sample holder for use with a core needle biopsy device, the core needle biopsy device including a piercer having a sample notch and a cutter movable relative to the sample notch to sever a tissue sample, the tissue sample holder comprising: a body defining a sample chamber, and a wiper catch; and a wiper movable relative to a portion of the tissue sample holder to manipulate a severed tissue sample from the sample notch of the piercer and into the sample chamber of the body, the wiper catch being configured to engage the wiper to store potential energy within the structure of the wiper.

### Example 17

The tissue sample holder of Example 16, further comprising a rotatable shaft, the wiper extending radially outwardly from the shaft, the body including an outer wall defining the sample chamber, the wiper being configured to rotate within the outer wall of the body to move a severed tissue sample within the sample chamber.

### Example 18

The tissue sample holder of Example 17, the wiper defining a curved portion, the curved portion defining a curvature oriented in a direction corresponding to a rotation direction of the shaft.

### Example 19

The tissue sample holder of any one or more of Examples 16 through 18, the wiper defining an engagement surface being configured to engage a severed tissue sample, the wiper including a plurality of ribs extending along a surface of the wiper opposite the engagement surface.

### Example 20

A method for collecting a tissue sample using a biopsy device, the method comprising: retracting a sample notch defined by a piercer proximally into a tissue sample holder; moving a wiper within the tissue sample holder to engage the wiper with a ledge; bending the wiper against the ledge to store potential energy within the wiper; and moving the wiper to disengage the wiper from the ledge and release the stored potential energy to propel a severed tissue sample into a sample chamber of the tissue sample holder.

### Example 21

The method of Example 20, wherein the act of moving the wiper includes rotating the wiper using a shaft coupled to the wiper.

### Example 22

The method of Example 21, the act of bending the wiper includes rotation the shaft coupled to the wiper.

### Example 23

The method of Examples 21 or 22, further comprising rotating the shaft 360° or more to collect a plurality of tissue samples.

### Example 24

The method of any one or more of Examples 20 through 23, further comprising moving the wiper to sweep the wiper across a sample catch of the tissue sample holder.

### Example 25

A biopsy device, comprising: a body defined by a probe and a holster; a needle assembly extending distally from the probe, the needle assembly being configured to sever a tissue sample; and a tissue sample holder having a body defining sample chamber, a drive member, and a wiper extending from a portion of the drive member, wherein the drive member is configured to move the wiper into engagement with a portion of the biopsy device to drive movement of the wiper at a variable rate to thereby propel a tissue sample from the wiper and into the sample chamber.

### Example 26

The biopsy device of Example 25, the drive member including a rotatable shaft, the wiper being secured to the shaft such that rotation of the shaft is configured to rotate the wiper relative to a portion of the needle assembly to thereby manipulate the tissue sample into the sample chamber.

### Example 27

The biopsy device of Examples 25 or 26, the needle assembly including a cutter and a piercer, the piercer being coaxially disposed within the cutter, the piercer including a notch configured to receive the tissue sample.

### Example 28

The biopsy device of Example 27, the wiper defining a length corresponding to a length of the notch of the piercer.

### Example 29

The biopsy device of any one or more of Examples 25 through 28, the wiper being configured for receipt within the notch of the piercer.

### Example 30

The biopsy device of any one or more of Examples 25 through 29, the wiper having a tissue engagement surface and a back surface opposite of the tissue engagement surface.

### Example 31

The biopsy device of Example 30, the wiper defining a curvature, the tissue engagement surface being associated with the curvature such that the tissue engagement surface defines a concavity.

### Example 32

The biopsy device of Examples 30 or 31, the tissue engagement surface having a hydrophobic coating.

### Example 33

The biopsy device of any one or more of Examples 30 through 32, the back surface having a plurality of ribs, the plurality of ribs extending from an inner end of the wiper to an outer end of the wiper.

### Example 34

The biopsy device of Example 33, each rib of the plurality of ribs defining an increasing thickness as each rib extends from the inner end to the outer end.

### Example 35

The biopsy device of any one or more of Examples 25 through 34, the wiper defining a thin substantially rectangular shape.

### Example 36

A core needle biopsy device, comprising: a body; a needle assembly extending distally from a portion of the body, the needle assembly including a piercer and a hollow cutter, the piercer including a sharp distal tip and a notch proximal to the distal tip, the piercer being slidably disposed within the cutter to sever a tissue sample into the notch of the piercer; a drive assembly configured to selectively move the piercer and the cutter; and a tissue sample holder having a cover, a driver, and a wiper, the cover being removably secured to a portion of the body, the cover defining a tissue sample chamber, the driver being configured to move the wiper relative to the cover to manipulate a severed tissue sample into the sample chamber defined by the cover.

### Example 37

The core needle biopsy device of Example 36, the cover of the tissue sample holder including a piercer interface, the piercer interface defining a portion of the tissue sample chamber.

### Example 38

The core needle biopsy device of Example 36, the cover of the tissue sample holder including an outer wall and a piercer interface, the piercer interface projecting inwardly at an angle from a portion of the outer wall to define a portion of the tissue sample chamber.

### Example 39

The core needle biopsy device of Example 36, the cover of the tissue sample holder including a piercer interface, the piercer interface defining a portion of the sample chamber, the piercer interface being configured to direct the severed tissue sample into the tissue sample chamber.

### Example 40

The core needle biopsy device of any of Examples 37 through 39, the piercer interface defining a ledge positioned proximate a portion of the piercer.

### Example 41

The core needle biopsy device of any of Examples 36 through 40, the cover including a sample catch projecting inwardly from a portion of the cover towards the tissue sample chamber.

### Example 42

The core needle biopsy device of any of Examples 36 through 40, the cover including a sample catch projecting inwardly from a portion of the cover towards the tissue sample chamber, the sample catch being configured to engage the wiper to scrape a tissue sample from the wiper.

### Example 43

The core needle biopsy device of any of Examples 36 and 41 through 42, the cover further including an outer wall, a proximal wall, and a piercer interface extending through an interior defined by the outer wall from the proximal wall, the outer wall, proximal wall, and piercer interface together defining the tissue sample chamber.

### Example 44

The core needle biopsy device of any of Examples 36 and 41 through 42, the cover further including an outer wall, a proximal wall, and a piercer interface extending through the outer wall from the proximal wall, the outer wall, proximal wall, and piercer interface together defining the tissue sample chamber, the outer wall defining a curved portion, the curved portion defining a portion of the tissue sample chamber, the piercer interface being disposed opposite of the curved portion.

### Example 45

The core needle biopsy device of any of Examples 36 and 41 through 42, the cover further including an outer wall, a proximal wall, and a piercer interface extending through the outer wall from the proximal wall, the outer wall, proximal wall, and piercer interface together defining the tissue sample chamber, the outer wall defining a curved portion, the curved portion defining a portion of the tissue sample chamber, the piercer interface being disposed opposite of the curved portion, a floor of the tissue sample chamber being defined by a lateral extension of the outer wall.

### Example 46

The core needle biopsy device of any of Examples 36 and 41 through 42, the cover further including an outer wall and a piercer interface, the outer wall and the piercer interface together defining a recessed portion corresponding to the tissue sample chamber.

### Example 47

The core needle biopsy device of any of Examples 36 through 46, the needle portion further including an end portion configured to drive movement of the cutter, the cover being configured to slidably receive the end portion.

### Example 48

The core needle biopsy device of any of Examples 36 through 47, the body defining a notch, the cover being configured to engage the body to enclose the notch.

### Example 49

The core needle biopsy device any of Examples 36 through 47, the body defining a notch, the cover being configured to form a snap-fit with the body to enclose the notch.

### Example 50

The core needle biopsy device of any of Examples 36 through 49, at least a portion of the cover being transparent.

### Example 51

A core needle biopsy device, comprising: a body defining a recessed portion; a needle assembly extending distally from a portion of the body, the needle assembly including a piercer and a hollow cutter, the piercer including a sharp distal tip and a notch proximal to the distal tip, the piercer being slidably disposed within the cutter to sever a tissue sample into the notch of the piercer; a drive assembly configured to selectively move the piercer and the cutter; a tissue extraction mechanism including a driver and a wiper, the driver being configured to move relative to the needle assembly to manipulate the tissue sample from the notch of the piercer; and a tissue collection assembly including a sample window, the sample window being configured to cover the recessed portion of the body, the sample window defining a sample chamber positioned proximate to the needle assembly, the sample chamber being configured to receive the tissue sample from the wiper.

### Example 52

The core needle biopsy device of Example 51, the sample window being removably coupled to the recessed portion of the body.

### Example 53

The core needle biopsy device of any of Example 51 or 52, further comprising one or more sealing elements disposed between the body and the sample window, the one or more sealing elements being configured to provide a seal between the sample window and the body.

### Example 54

The core needle biopsy device of any of Examples 51 through 53, a portion of the needle assembly being exposed within the recess such that the portion of the needle assembly is viable through the sample window.

### Example 55

A core needle biopsy device, comprising: a body defining a recessed portion; a needle assembly extending distally from a portion of the body and proximally through the recessed portion, the needle assembly including a piercer and a hollow cutter, the piercer including a sharp distal tip and a notch proximal to the distal tip, the piercer being slidably disposed within the cutter to sever a tissue sample into the notch of the piercer; a drive assembly configured to selectively move the piercer and the cutter; a tissue extraction mechanism including a driver and a wiper, the wiper projecting outwardly from a portion of the wiper; and a tissue collection assembly including a sample window, the sample window being configured to cover the recessed portion of the body, the sample window including a lateral projection, the lateral projection defining a sample chamber positioned proximate to the needle assembly, the driver of the tissue extraction mechanism being configured to rotate within the body to manipulate the tissue sample from the notch of the piercer and into the sample chamber.

### Example 56

The core needle biopsy device of any of Examples 36 through 50 and 55, the driver being configured to rotate the wiper relative to the cover.

### Example 57

The core needle biopsy device of any of Examples 36 through 50 and 55, the driver being configured to move the wiper laterally with respect to a longitudinal axis defined by the needle assembly.

### Examples 58

The core needle biopsy device of any of Examples 36 through 50 and 55 through 57, the driver being configured to move the wiper across the notch of the piercer toward the tissue sample chamber of the cover.

### Example 59

A method for collecting a tissue sample using a biopsy device, the method comprising: rotating a wiper relative to a sample notch defined by a piercer toward a cover defining a tissue sample chamber; wiping a first tissue sample from the notch of the piercer into the tissue sample chamber such that the first tissue sample is held in the tissue sample chamber; and removing the cover from the biopsy device with the first tissue sample disposed within the tissue sample chamber.

### Example 60

The method of Example 59, further comprising wiping a second tissue sample from the notch of the piercer into the tissue sample chamber to contain both the first tissue sample and the second tissue sample within the tissue sample chamber.

### Example 61

The method of Example 59, further including the step of collecting the first tissue sample and the second tissue sample from the tissue sample chamber after the step of removing the cover.

### Example 62

The method of any of Examples 59 through 61, the step of rotating the wiper includes moving the wiper laterally relative to a longitudinal axis defined by the piercer.

### Example 63

The method of any of Examples 59 through 62, the step of wiping the first tissue sample including propelling the first tissue sample from the wiper and into the tissue sample chamber of the cover.

### Example 64

The method of any of Examples 59 through 62, the step of wiping the first tissue sample including scraping a portion of the wiper across a sample catch defined by the cover.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

It should be understood that any of the versions of instruments described herein may include various other features in addition to or in lieu of those described above. By way of example only, any of the instruments described herein may also include one or more of the various features disclosed in any of the various references that are incorporated by reference herein. It should also be understood that the teachings herein may be readily applied to any of the instruments described in any of the other references cited herein, such that the teachings herein may be readily combined with the teachings of any of the references cited herein in numerous ways. Other types of instruments into which the teachings herein may be incorporated will be apparent to those of ordinary skill in the art.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

1. A core needle biopsy device, comprising:
a needle assembly, wherein the needle assembly includes a piercer and a hollow cutter, wherein the piercer includes a sharp distal tip and a notch proximal to the distal tip, wherein the piercer is slidably disposed within the cutter to sever a tissue sample into the notch of the piercer;
a drive assembly configured to selectively move the piercer and the cutter; and
a tissue sample holder having a sample chamber, a driver, and a wiper, the driver being configured to bend the wiper against a portion of the biopsy device to manipulate a severed tissue sample into the sample chamber.

2. The core needle biopsy device of claim 1, the driver including a rotatable shaft, the wiper being secured to the shaft such that rotation of the shaft is configured to rotate the wiper relative to the piercer to thereby manipulate a severed tissue sample into the sample chamber.

3. The core needle biopsy device of claim 1, the needle assembly further comprising a cutter end portion associated with a proximal end of the cutter, the cutter end portion including a ledge configured to engage the wiper.

4. The core needle biopsy device of claim 3, the cutter end portion including a curved surface extending towards a portion of the piercer.

5. The core needle biopsy device of claim 1, the tissue sample holder further including an outer cover configured to removably couple to a housing of the biopsy device, the outer cover defining at least a portion of the sample chamber.

6. The core needle biopsy device of claim 5, the outer cover including one or more wall defining the sample chamber.

7. The core needle biopsy device of claim 5, the outer cover including a piercer interface positioned proximate a portion of the piercer.

8. The core needle biopsy device of any one or more of claim 5, the outer cover including a sample catch, the sample catch being configured to engage a portion of the wiper to scrape a tissue sample from the wiper.

9. The core needle biopsy device of claim 8, the sample catch being positioned proximate the sample chamber.

10. The core needle biopsy device of claim 5, the outer cover including one or more connectors configured to releasably secure the outer cover to the housing of the biopsy device.

11. The core needle biopsy device of claim 1, the tissue sample holder including a single wiper.

12. The core needle biopsy device of claim 1, the wiper including a material having a durometer of 76.5 to 93.5.

13. The core needle biopsy device of claim 1, the wiper including a material having a durometer of about 85.

14. The core needle biopsy device of claim 1, the wiper having a hydrophobic coating.

15. The core needle biopsy device of claim 1, further comprising a body having a distal end, the needle assembly extending distally from the distal end of the body, the tissue sample holder being disposed on the distal end of the body.

16. A tissue sample holder for use with a core needle biopsy device, the core needle biopsy device including a piercer having a sample notch and a cutter movable relative to the sample notch to sever a tissue sample, the tissue sample holder comprising:
a body defining a sample chamber, and a wiper catch; and
a wiper movable relative to a portion of the tissue sample holder to manipulate a severed tissue sample from the sample notch of the piercer and into the sample chamber of the body, the wiper catch being configured to engage the wiper to store potential energy within the structure of the wiper.

17. The tissue sample holder of claim 16, further comprising a rotatable shaft, the wiper extending radially outwardly from the shaft, the body including an outer wall defining the sample chamber, the wiper being configured to rotate within the outer wall of the body to move a severed tissue sample within the sample chamber.

18. The tissue sample holder of claim 17, the wiper defining a curved portion, the curved portion defining a curvature oriented in a direction corresponding to a rotation direction of the shaft.

19. The tissue sample holder of claim 16, the wiper defining an engagement surface being configured to engage a severed tissue sample, the wiper including a plurality of ribs extending along a surface of the wiper opposite the engagement surface.

20. A method for collecting a tissue sample using a biopsy device, the method comprising:
retracting a sample notch defined by a piercer proximally into a tissue sample holder;
moving a wiper within the tissue sample holder to engage the wiper with a ledge;
bending the wiper against the ledge to store potential energy within the wiper; and
moving the wiper to disengage the wiper from the ledge and release the stored potential energy to propel a severed tissue sample into a sample chamber of the tissue sample holder.

21.-64. (canceled)
